(19) United States
(12) Patent Application Publication
(10) Pub. No.: US 2003/0031720 A1
(43) Pub. Date: Feb. 13, 2003

(54) Method for producing pharmaceutical dosage forms

(76) Inventors: Tobias Laich, Koln (DE); Carola Pörtner, Rosrath (DE); Jan-Olav Henck, Willich (DE)

Correspondence Address: JEFFREY M. GREENMAN, VICE PRESIDENT, PATENTS AND LICENSING, BAYER CORPORATION, 400 MORGAN LANE, WEST HAVEN, CT, 06516, US

(21) Appl. No.: 10/204,837

(22) PCT Filed: Feb. 13, 2001
(86) PCT No.: PCT/EP01/01565

(51) Int. Cl. A61K 31/401; B29B 9/00; A61K 31/366; A61K 31/225; A61K 9/14

(52) U.S. Cl. 424/489; 264/5; 514/423; 514/460; 514/548

(57) ABSTRACT

The invention relates to a method for producing a granulate while using spray-dried D-mannitol and to the production of pharmaceutical dosage forms comprised of granulates of this type. The invention additionally relates to granulates obtained by using this method and to pharmaceutical dosage forms, which contain statins, especially cerivastatin, and which can be produced from said granulates.

The present invention relates to a method for producing granules by use of spray-dried D-mannitol, and to the production of phararmaceutical dosage forms from such granules. The invention further relates to granules obtainable by this method and to pharmaceutical dosage forms which can be produced therefrom and comprise active pharmaceutical ingredients, in particular statins.

WO 97/38960 describes D-mannitol which has good properties as filler for the production of pharmaceutical preprarations, in particular tablets. The D-mannitol described therein consists of a mixture of crystals in the δ form (modification III) and the β form (modification I). It is also described therein that this D-mannitol is suitable inter alia for producing solid pharmaceutical preparations of cerivastatin.

WO 98/57917 describes a method for producing medicaments comprising HMG-CoA reductase inhibitors. A preferred embodiment described therein is the production of cerivastatin-containing granules by wet granulation with mannitol.

It is additionally known that spray-dried mannitol can be employed as filler in direct tableting. Thus, U.S. Pat. No. 3,145,146 describes the production of spray-dried D-mannitol and its use for direct tableting. In addition, for example, U.S. Pat. No. 5,958,471 describes preparations and compacted articles which comprise a mixture of spray-dried polyols, including mannitol.

It has now surprisingly been found in further development of the method described in WO 98/57917 that excellent results are obtained when spray-dried D-mannitol is employed in the granulation. This finding is surprising in particular because spray-dried D-mannitol is normally employed as filler for direct tableting, i.e. all the components of the relevant tablets are mixed dry and then compressed to tablets. The skilled worker would expect that the advantageous properties of spray-dried mannitol would be lost on processing of spray-dried inannitol under moist conditions. Unexpectedly, however, this is not the case in the method of the invention.

The invention relates to a method for producing granules in which

(a) a solution or suspension which comprises an active pharmaceutical ingredient and, where appropriate, comprises other binders and/or excipients is granulated with spray-dried mannitol and, where appropriate, other binders and/or excipients and

(b) the resulting granules are dried.

The invention further relates to granules comprising a statin and spray-dried D-mannitol.

The invention further relates to a method for producing a pharmaceutical dosage form, in which the granules described above are converted, where appropriate with the addition of other excipients, into the desired dosage form.

The invention further relates to a pharmaceutical dosage form comprising a statin and spray-dried D-mannitol.

The invention further relates to the use of spray-dried mannitol for producing pharmaceutical dosage forms comprising a statin.

Solvents suitable for the solution or suspension comprising the active pharmaceutical ingredient in the method of the invention for producing granules are water, alcohols such as methanol, ethanol, isopropanol, n-propanol and other volatile solvents such as dichloromethane, acetone, ethyl acetate or other pharmaceutically acceptable solvents. It is also possible to employ mixtures of the aforementioned solvents. Hydrous solvents or solvent mixtures are preferred; water is particularly preferred.

Other binders suitable for the solution or suspension comprising the active pharmaceutical ingredient are all conventional pharmaceutically acceptable binders; examples are polyvinylpyrrolidones, gelatin, starch derivatives and cellulose derivatives (natural or synthetic) such as, for example, hydroxypropyl-methylcellulose, methylcellulose, hydroxypropylcellulose, methylstarch, pregelatinized starch, dextrins, but also dextrans, alginates or derivatives thereof.

Preference is given to polyvinylpyrrolidones such as, for example, Kollidon® 25.

Other excipients which can be employed are all conventional pharmaceutical excipients, for example as fillers—apart from spray-dried D-mannitol—celluloses and derivatives thereof (e.g. microcrystalline cellulose, native cellulose, hydroxy-propylcellulose, hydropropylmethylcellulose, methylcellulose), sugars (e.g. lactose, fructose, sucrose, glucose, maltose), other sugar alcohols (e.g. sorbitol, xylitol, lactitol), inorganic fillers (e.g. calcium phosphates, calcium sulfates), starches and derivatives thereof (corn starch, potato starch, wheat starch, dextrins, pregelatinized starches) and all other excipients required to produce pharmaceutical formulations of the desired properties, e.g. lubricants (e.g. magnesium stearate, calcium stearate, calcium behenate, sodium stearyl fumarate), e.g. disintegration aids (“disintegrants” e.g. crosslinked polyvinylpyrrolidone, sodium carboxymethylcellulose, sodium carboxymethylstarch, starches), e.g. wetting agents (e.g. sodium lauryl sulfate, polyoxyethylene sorbitan fatty acid esters, sorbitan fatty acid esters, stearic acid, lecithins), e.g. alkaline additives (e.g. sodium hydroxide, potassium hydroxide, amines, ammonia, calcium hydroxide, magnesium hydroxide), e.g. stabilizers (antioxidants such as, for example, ascorbic acid, butylated hydroxytoluene (BHT), butylated hydroxyanisole (BHA), tocopherols, citric acid, EDTA sodium) e.g. aromas, e.g. colored pigments or coloring agents.

The proportion of binder in the complete mixture is preferably 0 to 20% (m/m). The proportion of fillers and excipients in the complete mixture is 20 to 99%, preferably 50 to 99%, particularly preferably 70 to 99% (m/m).

If the proportion of fillers and excipients is considered to be 100%, then the proportion of spray-dried D-mannitol is 50 to 100%, preferably 60 to 100%, particularly preferably 70 to 100% (m/m).

The temperature at which the solvent-containing granules are dried is generally 40 to 120° C., preferably 60 to 100° C. (temperature of the drying medium). Step (a) of the method of the invention (the granulation) can preferably be carried out for example in a high-shear mixer.

Spray-dried D-mannitol is employed according to the invention as essential filler. Spray-dried D-mannitol is distinguished by the D-mannitol therein being present in modifications I (β form) and II (α form). Moreover, the proportion of modification III (δ form) in spray-dried mannitol is usually less than 5% (m/m). The D-mannitol employed according to the invention has an average particle size of from 5 to 400 μm, preferably 50 to 350 μm, particularly preferably 100 to 250 μm.

The individual particles of the spray-dried mannitol (granule particles) have a particle size distribution whose median (x50) is located in the stated ranges. This is based on a volume distribution, and all particles are assumed to be spherical.

Such a measurement can be determined by laser light diffraction such as, for example, by means of a Sympatec HELOS laser diffraction instrument with focal length R5 (500 mm) using the SUCELL wet dispersing unit with integrated ultrasonic bath (35 kHz, 50 W) (Baysilon M 10 oil is used as dispersing medium in this case), and the sample is treated with ultrasound for 3 min before measurement. The Sympatec WINDOX software is used to analyze the measurement.

In contrast to spray-dried mannitol, the D-mannitol described in WO 97/38960 is in the form of modifications I and III. Modification II is virtually undetectable in this mannitol (proportion ≦5% (m/m)).

The method of the invention is suitable in principle for all active pharmaceutical ingredients which are not changed in an unwanted manner under the conditions of the method. “Active pharmaceutical ingredients” are intended here to mean substances which may display a large physiological effect if present or supplied in relatively small amounts. The term is intended to mean in particular medicinally active substances (“drugs, medicinal substances”) which are suitable for the prophylaxis, cure or alleviation of disorders. Statins in particular are employed as active pharmaceutical ingredients. Statins are a class of HMG-CoA reductase inhibitors with the following formula

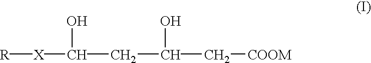

in which

R is an organic radical,

X is a group —CH₂—CH₂— or —CH═CH—; in particular in the (E) form, and

M is a physiologically acceptable cation, for example from the series of alkali metal cations, preferably sodium or potassium, and is an ammonium ion.

Apart from the open-chain salt form depicted in formula (I), the statins may also be employed in the form of their δ-lactone.

The statins which are in turn particularly preferred according to the invention are

atorvastatin (commercially available under the name Lipitor® from Parke-Davis);

cerivastatin (commercially available under the name Lipobay® or Baycol® from Bayer);

fluvastatin (commercially available under the name Lescol® from Novartis);

lovastatin (commercially available under the name Mevacor® from Merck);

pravastatin (commercially available under the name Lipostat® from Bristol-Myers Squibb);

simvastatin (commercially available under the name Zocor® from Merck);

itavastatin (also called “nisvastatin”; NK-104; systematic name: [S-[R*,S*-(E)]]-7-[2-cyclopropyl-4-(4-fluorophenyl)-3-quinolinyl]-3,5-dihydroxy-6-heptenoic acid);

dalvastatin;

mevastatin;

dihydrocompactin;

compactin; and

S-4522; systematic name (+)-(3R,5S)-bis-(7-(4-(4-fluorophenyl)-6-isopropyl-2-(N-methyl-N-methanesulfonylamino)-pyrimidin-5-yl)-3,5-dihydroxy-6(E)-heptenoic acid;

and their respective salts, hydrates, alcoholates, esters, lactones and tautomers, with very particular preference among these for atorvastatin, cerivastatin, fluvastatin, lovastatin, pravastatin, itavastatin, simvastatin and (+)-(3R,5S)-bis-(7-(4-(4-fluorophenyl)-6-isopropyl-2-(N-methyl-N-methanesulfonylamino)pyrimidin-5-yl) -3,5-dihydroxy-6(E)-heptenoic acid and their respective salts, hydrates, alcoholates, esters, lactones and tautomers.

Among these in turn very particular preference is given to cerivastatin and atorvastatin and their respective salts, hydrates, alcoholates, esters, lactones and tautomers.

For further details concerning the aforementioned statins, reference is made to the discussions in Drugs of the Future 1994, 19(6), pages 537-541 and 1995, 20(6), page 611 and 1996, 21(6), page 642, the full contents of each of which is incorporated herein by reference.

The term “salt” for the purpose of the present invention means in each case physiologically acceptable salts of the respective compounds: these may be, for example, may be salts with mineral acids, carboxylic acids or sulfonic acids, in particular with hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, citric acid, fumaric acid, maleic acid or benzoic acid or else mixed salts thereof. However, salts with conventional bases are also possible, such as, for example, alkali metal salts (e.g. sodium or potassium salts), alkaline earth metal salts (e.g. calcium or magnesium salts) or ammonium salts, derived from ammonia or organic amines such as, for example, diethylamine, triethylamine, ethyldiisopropylamine, procaine, dibenzylamine, N-methylmorpholine, dihydroabietylamine, 1-ephenamine or methylpiperidine and mixed salts thereof.

The statins preferably employed for the purposes of this invention are in the form of their salts.

Examples of statin salts which can be used according to the invention are the monosodium salt of fluvastatin; the monopotassium salt and the calcium salt of itavastatin; and the calcium salt of (+)-(3R,5S)-bis-(7-(4-(4-fluorophenyl)-6-iso-propyl-2-(N-methyl-N-methanesulfonylamino)pyrimidin-5-yl)-3,5-dihydroxy-6(E)-heptenoic acid (“ZD 4522” or “S 4522” respectively from Shionogi and AstraZeneca). Further examples of statin salts which can be used according to the invention are the monosodium and monopotassium salts, and the magnesium and calcium salts of cerivastatin, of atorvastatin and of pravastatin. The cerivastatin salts, especially the sodium salt (also referred to as cerivastatin sodium) are particularly preferably employed.

Further preferred HMG-CoA reductase inhibitors are described in EP-A-0 325 130 and in EP-A-0-491 226, both in the name of Bayer AG, the contents of which is incorporated herein by reference. EP-A-0 325 130 relates to substituted pyridines, and EP-A-0-491 226 describes substituted pyridyldihydroxyheptenoic acid derivatives and their salts, including in particular the cerivastatin which is particularly preferred according to the invention (claim 6 of EP-A-0 491 226).

Equally preferred according to the invention are the HMG-CoA reductase inhibitors mentioned in the publication Bioorganic & Medicinal Chemistry, Vol. 5, No. 2, pages 437-444 (1997), the full disclosure of which is incorporated herein by reference.

Another review of HMG-CoA reductase inhibitors is present in Pharmazie in unserer Zeit, Vol. 28, No. 3, pages 147-152 (1999).

To produce the active ingredient-containing solution or suspension it proves beneficial in the case of the statins in open-chain salt form, in particular for cerivastatin sodium, first to produce the actual active ingredient from a suitable active ingredient precursor, the ester or, in particular, the lactone, by treatment with aqueous base, in particular with an essentially equivalent molar amount, to add to this so-called hydrolysis solution the binder (preferably, for example, PVP) in the form of an aqueous solution, and to add to this mixture where appropriate another solution comprising excipients, in particular, for example, an aqueous solution of a base (e.g. sodium hydroxide). The mixture obtained in this way can then first be granulated with spray-dried mannitol by the method of the invention and then be dried.

“Granules” are intended here to mean a collection of granule particles; a granule particle in turn is an aggregate of powder particles (whole crystals, crystal fragments, etc.). Granule particles typically have an irregular surface and a porous structure. “Granulation” means the conversion of powder particles into granule particles.

The granules of the ivention comprising a statin and spray-dried mannitol are preferably produced by the method of the invention. They are distinguished by advantageous properties: thus, they show an excellent flowability, which improves the meterability of the granules and facilitates processing in the production of pharmaceutical dosage forms, e.g. in tableting. The granules of the invention also show compaction properties. Finally, the fines content of the granules of the invention is markedly reduced, which means that less dust is evolved. This has safety advantages (less dust contamination of operatives) and leads to less expenditure on cleaning.

The pharmaceutical dosage forms of the invention comprising a statin and spray-dried D-mannitol can be produced by methods known per se. The granules of the invention are preferably employed for their production.

Suitable pharmaceutical dosage forms are known to the skilled worker. Examples which may be mentioned are sacchets, capsules and tablets. The granules are preferably processed to tablets. In the production of the pharmaceutical dosage forms it is also possible to add suitable excipients such as, for example, the above-mentioned fillers, lubricants, disintegration aids, wetting agents, aromas, coloring agents, stabilizers etc. If desired, the resulting tablets can be provided with a suitable coating in a conventional way. The method steps necessary for this are known to the skilled worker. Examples of suitable coatings are natural, synthetic or semisynthetic polymers (shellac, hydroxypropylmethylcellulose, polymethacrylates, cellulose acetate) or else starch syrups in combination with sugars (sucrose, glucose, fructose etc.) together with coloring agents or pigments. Hydroxypropylmethylcellulose is preferably used in combination with iron oxides and/or titanium dioxide.

The methods described above for producing granules and pharmaceutical dosage forms are particularly suitable when the active ingredient is employed in only very small amounts, e.g. less than 5%, preferably less than 1% (proportion by weight in the final formulation). It is possible by further processing of the active ingredient solution or suspension to give the granulation liquid and subsequent coating or granuation of the filler or filler mixture to produce pharmaceutical preparations which are distinguished by excellent uniformity of active ingredient distribution. The generally known problems arising on convention (dry) mixing of components with very different proportions in a complete mixture are thus avoided in a simple manner.

### EXAMPLES

### Example 1

0.4 mg cerivastatin dosage

5228.13 g of D-mannitol spray-dried (Pearlitol 200 SD, Roquette, France)

25.00 g of cerivastatin sodium (from cerivastatin lactone)

8.12 g of sodium hydroxide (about 5.97 g remain in the granules)

112.50 g of polyvinylpyrolidone (Kollidon 25, BASF, Germany)

437.50 g of water

22.92 g of cerivastatin lactone are reacted with 233.91 g of water and 2.12 g of NaOH to give cerivastatin sodium solution (hydrolysis solution). A solution is prepared from PVP, the remaining amount of water and the remaining amount of NaOH. This solution is mixed with the hydrolysis solution and used as liquid for the granulation. The spray-dried D-mannitol is introduced into a high-shear mixer (MGT 30, Lödige, Germany) and premixed at 200 rpm (chopper stage 1) for 1 min. The granulation liquid is added at a constant rate in 7 min. Granulation is then continued for a further minute. The mixer is emptied through a 4 mm grater/shredder (Alexanderwerk, Germany). The resulting granules are dried in a fluidized bed (Glatt, Switzerland) at an inlet air temperature of 70° C. until the product temperature is 41.5° C.

The dry granules are mixed with 3% (m/m) crosslinked PVP (Polyplasdone XL, BASF, Germany) and 2% (m/m) magnesium stearate (Greven, Germany) for 5 min. This is followed by compression to tablets weighing 90 mg (format 6 mm WR 9 mm) round tablets.

The tablets obtained in this way can also be coated.

### Example 2

0.8 mg cerivastatin dosage

5228.13 g of D-mannitol spray-dried (Pearlitol 200 SD, Roquette, France)

25.00 g of cerivastatin sodium (from cerivastatin lactone)

8.12 g of sodium hydroxide (about 5.97 g remain in the granules)

112.50 g of polyvinylpyrolidone (Kollidon 25, BASF, Germany)

437.50 g of water

22.92 g of cerivastatin lactone are reacted with 233.91 g of water and 2.12 g of NaOH to give cerivastatin sodium solution (hydrolysis solution). Another solution is produced from PVP and 178.60 g of water. This is mixed with the hydrolysis solution. A solution is prepared from the remaining amount of water and the remaining amount of NaOH. This solution is mixed with the previously produced mixture of hydrolysis solution and PVP solution and used as liquid for the granulation. The spray-dried mannitol is introduced into a high-shear mixer (MGT 30, Lödige, Germany) and premixed at 200 rpm (chopper stage 1) for 1 min. The granulation liquid is added at a constant rate in 7 min. Granulation is then continued for a further minute. The mixer is emptied through a 4 mm grater/shredder (Alexanderwerk, Germany). The resulting granules are dried in a fluidized bed (Glatt, Switzerland) at an inlet air temperature of 70° C. until the product temperature is 41.5° C.

The dry granules are mixed with 3% (m/m) crosslinked PVP (Polyplasdone XL, BASF, Germany) and 2% (m/m) magnesium stearate (Greven, Germany) for 5 min. This is followed by compression to tablets weighing 180 mg (format 8 mm WR 12 mm) round tablets.

The tablets obtained in this way can also be coated.

### Example 3

0.2 mg cerivastatin dosage

5240.63 g of D-mannitol spray-dried (Pearlitol 200 SD, Roquette, France)

12.50 g of cerivastatin sodium (from cerivastatin lactone)

7.06 g of sodium hydroxide (about 5.97 g remain in the granules)

112.50 g of polyvinylpyrolidone (Kollidon 25, BASF, Germany)

437.50 g of water

11.46 g of cerivastatin lactone are reacted with 116.95 g of water and 1.06 g of NaOH to give cerivastatin sodium solution (hydrolysis solution). A solution is prepared from PVP, the remaining amount of water and the remaining amount of NaOH. This solution is mixed with the hydrolysis solution and used as liquid for the granulation. The spray-dried mannitol is introduced into a high-shear mixer (MGT 30, L\ddot{o}dige, Germany) and premixed at 200 rpm (chopper stage 1) for 1 min. The granulation liquid is added at a constant rate in 7 min. Granulation is then continued for a further minute. The mixer is emptied through a 4 mm grater/shredder (Alexanderwerk, Germany). The resulting granules are dried in a fluidized bed (Glatt, Switzerland) at an inlet air temperature of 70° C. until the product temperature is 41.5° C.

The dry granules are mixed with 3% (m/m) crosslinked PVP (Polyplasdone XL, BASF, Germany) and 2% (m/m) magnesium stearate (Greven, Germany) for 5 min. This is followed by compression to tablets weighing 90 mg (format 6 mm WR 9 mm) round tablets.

The tablets obtained in this way can also be coated.

### Example 4

0.1 mg cerivastatin dosage

5246.88 g of D-mannitol spray-dried (Pearlitol 200 SD, Roquette, France)

6.25 g of cerivastatin sodium (from cerivastatin lactone)

6.53 g of sodium hydroxide (about 5.97 g remain in the granules)

112.50 g of polyvinylpyrolidone (Kollidon 25, BASF, Germany)

437.50 g of water

5.73 g of cerivastatin lactone are reacted with 58.47 g of water and 0.53 g of NaOH to give cerivastatin sodium solution (hydrolysis solution). A solution is prepared from PVP, the remaining amount of water and the remaining amount of NaOH. This solution is mixed with the hydrolysis solution and used as liquid for the granulation. The spray-dried mannitol is introduced into a high-shear mixer (MGT 30, Lödige, Germany) and premixed at 200 rpm (chopper stage 1) for 1 min. The granulation liquid is added at a constant rate in 7 min. Granulation is then continued for a further minute. The mixer is emptied through a 4 mm grater/shredder (Alexanderwerk, Germany). The resulting granules are dried in a fluidized bed (Glatt, Switzerland) at an inlet air temperature of 70° C. until the product temperature is 41.5° C.

The dry granules are mixed with 3% (m/m) crosslinked PVP (Polyplasdone XL, BASF, Germany) and 2% (m/m) magnesium stearate (Greven, Germany) for 5 min. This is followed by compression to tablets weighing 90 mg (format 6 mm WR 9 mm) round tablets.

The tablets obtained in this way can also be coated.

### Example 5

As example 1 but time for addition of granulation liquid 2 min.

### Example 6

As example 1 but time for addition of granulation liquid 3 min.

### Example 7

As example 1 but time for addition of granulation liquid 5 min.

1. A method for producing granules in which
(a) a solution or suspension which comprises an active pharmaceutical ingredient and, where appropriate, comprises other binders and/or excipients is granulated with spray-dried D-mannitol and, where appropriate, other binders and/or excipients and
(b) the resulting granules are dried.

2. A method as claimed in claim 1, where the active pharmaceutical ingredient is a statin.

3. A method as claimed in claim 2, wherein the statin is lovastatin, simvastatin, pravastatin, fluvastatin, atorvastatin, cerivastatin, itavastatin or S-4522.

4. Granules comprising a statin and spray-dried D-mannitol

5. Granules comprising cerivastatin and spray-dried D-mannitol

6. A method for producing a pharmaceutical dosage form, in which the granules as claimed in either of claims 4 or 5 are converted, where appropriate with the addition of other excipients, into the desired dosage form.

7. A method as claimed in claim 6, in which the granules are produced as claimed in claim 1.

8. A method as claimed in either of claims 6 or 7, in which a tablet is produced from the dried granules.

9. A pharmaceutical dosage form comprising a statin and spray-dried D-mannitol.

10. A pharmaceutical dosage form comprising cerivastatin and spray-dried D-mannitol.

11. A pharmaceutical dosage form as claimed in claim 9 or 10 in the form of tablets

12. The use of spray-dried D-mannitol for producing pharmaceutical dosage forms comprising a statin.

13. The use as claimed in claim 12, where the statin is cerivastatin.
